(19) United States
(12) Patent Application Publication
(10) Pub. No.: US 2011/0319833 A1
(43) Pub. Date: Dec. 29, 2011

(54) PROTECTIVE GUARD FOR NEEDLES OF INJECTION DEVICES

(76) Inventor: THOMAS CHUN, Northridge, CA (US)

(21) Appl. No.: 12/822,717

(22) Filed: Jun. 24, 2010

(51) Int. Cl. A61M 5/32

(52) U.S. Cl. 604/198

(57) ABSTRACT

A self-deploying cover for protection against unintended pricks from injection devices. The cover comprises a base which engages the injection device, and a protective sleeve which covers the sharp point and moves relative to the base to expose and cover the sharp point. The sleeve is guided by a projection which rides within a generally V-shaped groove formed in the base. The groove comprises deflectable arms which deflect to pass the projection, but which oppose return of the projection after passing. The sleeve is spring urged to the extended position and is automatically withdrawn during injections. After an injection, the sleeve is locked into the extended position by the last deflectable arm.

### CROSS-REFERENCE TO RELATED APPLICATION

This application claims the benefit of the filing date under 35 USC 119(e) of the filing date of U.S. Provisional application Ser. No. 12/466,118, filed May 14, 2009, the contents of which are incorporated herein by reference.

### BACKGROUND OF INVENTION

The present invention pertains to injection devices such as syringes and autoinjectors, and more particularly to a guard arrangement which covers sharp points of injection devices.

### SUMMARY

Syringes, autoinjectors, and like injection devices have long been used to deliver medicaments and other substances transdermally. An injection device typically has a reservoir for storing a liquid to be injected, a plunger to pressurize the stored liquid, and an elongated slender pointed needle for penetrating skin and other tissues, so as to deliver the pressurized liquid into the body.

Because the point is very sharp, it easily penetrates human tissues, clothing, protective gear such as rubber gloves, and other articles. This presents a hazard to medical personnel due to unintended pricking of the personnel's body. This can be uncomfortable, can cause the personnel to drop or otherwise mishandle equipment, and most of all, threatens to transfer contaminants from an injected person's blood to the medical personnel.

Countermeasures directed to unintended pricking of the body have been proposed. However, many countermeasures introduce annoying drawbacks. For example, a cover may need to be manually installed and manually removed when needed. This may for example oblige medical personnel to put down other equipment and objects and perform the necessary installation or removal, or otherwise interfere with expeditious performance of medical tasks.

There remains a need for an uncomplicated yet effective protective device for preventing unintended pricking due to exposed needles, or “sharps”, as they have become informally known, which is self-deploying and which makes minimal demands on medical personnel using injection devices.

The present invention addresses the above concern by providing a self-deploying cover assembly for exposed needles and the like. The self-deploying cover may be a self-contained device which is installable to the needle assembly of an injection device such as a syringe or autoinjector having a separately installable needle assembly. The operative principle includes a guide structure, for example comprising a guiding groove formed in a first part, and a projection which is formed in a relatively movable second part, and which rides within the groove.

In a preferred embodiment, the projection is constrained from returning to a prior location within the groove by several unidirectional gates.

The self-deploying cover not only provides the safety feature of preventing unintended pricking by a sharp point, but also can control depth of insertion of the needle.

It is possible to provide the protective cover in various dimensions corresponding to standard syringe or needle dimensions.

The protective device may comprise a mobile cover which moves relatively with respect to the needle. The protective device may comprise a sleeve (referred to above as the second part) which is axially slidable along the injection device. The sleeve is mobile, engaging a fixed or stationary part or base of the cover assembly (the base is referred to above as the first part). The stationary part engages the injection device and guides the movable sleeve. A spring urges the sleeve into the extended position where it will cover the sharp point, thereby providing protection. Prior to use, the sleeve is readily retractable in relationship to the stationary base. The act of injection causes the sleeve to retract, thereby enabling injection. After injection, as the injection device is withdrawn, the sleeve extends to the protective position, where it locks. Locking can be manually overcome, for example, in preparation of a subsequent injection, but is sufficiently robust as to resist casual and unintended retraction which would leave the sharp point exposed.

The novel protective cover may include a self-closing resilient membrane which further covers the point, thereby assisting in excluding contaminants from contact with the needle when the latter is not in use.

In a still further option, the novel protective cover may be furnished with a frangible outer cover which keeps the sleeve sterile prior to the first use. The frangible outer cover may be manually detached and discarded in preparation for an injection.

### BRIEF DESCRIPTION OF THE DRAWINGS

The drawings, when considered in connection with the following description, are presented for the purpose of facilitating an understanding of the subject matter sought to be protected.

FIGS. 1-23B provide background for in depth understanding the invention, and duplicate subject matter of the above referenced prior application. FIGS. 24-32 illustrate how the principles of the prior application are adapted to the present invention.

FIG. 1 is an exploded side view of a syringe shown axially aligned with a separate protective cover according to at least one aspect of the invention.

FIG. 2 is a side view of the components of FIG. 1 shown assembled.

FIG. 3 is a diagrammatic detail view showing guiding structure formed as part of the components of FIG. 1, and illustrating a first or initial position of two mutually movable parts.

FIG. 4 is an end view of the component seen at the right of FIG. 3.

FIG. 5 is similar to FIG. 3, but shows an intermediate position of the two mutually movable parts, and also shows how in the intermediate position an exposed needle point.

FIG. 6 is similar to FIG. 3, and shows a final position of the two mutually movable parts.

FIG. 7 is an enlarged side detail view of a guideway formed in one of the components of FIG. 3.

FIG. 8 is a cross sectional side view of the components of FIG. 3.

FIG. 9 is a cross sectional side view of the components of the protective cover as seen in FIG. 5, with the needle point shown in FIG. 5 deleted from the view of FIG. 9.

FIG. 10 is a cross sectional side view of the component seen at the right in FIG. 8, illustrating an optional sealing feature.

FIG. 11 is a partly cross sectional side view of the components of FIG. 3, showing an optional discardable protective cap.

FIG. 12 is a perspective exploded detail view of guide structure shown in FIG. 3, showing an additional component according to a further aspect of the invention.

FIG. 13 is an end view of the components of FIG. 12 shown assembled and drawn to reduced scale.

FIG. 14 is a diagrammatic side view of an injection device incorporating a retractable needle, shown in an initial position which would appear prior to an injection, and shown partially in cross section.

FIG. 15 is similar to FIG. 14, but shows the plunger and needle moved to an injected position.

FIG. 16 is similar to FIG. 14, but shows the plunger and needle returned to the retracted position by a spring.

FIG. 17 is a diagrammatic side view of an injection device which incorporates the protective features of the arrangement of FIG. 1 integrally into the injection device, rather than comprising separate components as depicted in FIG. 1.

FIG. 18 is a diagrammatic representation of an alternative to the groove arrangement seen in FIG. 7, showing deflection that occurs as parts move.

FIG. 19 is similar to FIG. 18, but shows a subsequent stage of events wherein entrapment has occurred.

FIG. 20A is a diagrammatic side cross sectional view of a syringe according to a further aspect of the invention, showing an initial condition which exists just prior to an injection.

FIG. 20B is a diagrammatic representation of the components of FIG. 18, showing relationship between events occurring in the entrapment feature of FIG. 18 and events depicted in FIG. 20A.

FIG. 21A is similar to FIG. 20A, but shows a second stage of events out of four stages.

FIG. 21B is similar to FIG. 20B, but reflects the second stage of events shown in FIG. 21A.

FIG. 22A is similar to FIG. 20A, but shows a third stage of events of the four stages.

FIG. 22B is similar to FIG. 20B, but reflects the third stage of events shown in FIG. 22A.

FIG. 23A is similar to FIG. 20A, but shows the fourth and final stage of events.

FIG. 23B is similar to FIG. 20B, but reflects the fourth and final stage of events shown in FIG. 23A.

FIG. 24 is an exploded side view of three of the novel components, shown with two conventional components of an injector device.

FIG. 25 is an exploded side view of the components of FIG. 24, and also introduces an additional novel component and one additional conventional component of an injector device.

FIG. 26 is a side detail view illustrating an operating principle of one of the novel components seen towards the center of FIG. 24.

FIG. 27 is a diagrammatic side view showing the components of FIG. 25 fully assembled, with the conventional component seen at the left of FIG. 25 having been removed and discarded.

FIG. 28 is a diagrammatic side view depicting the components of FIG. 27, but shows projection of a needle of the injection device in preparation for an injection.

FIG. 29 is similar to FIG. 28, but illustrates still further projection of the needle.

FIG. 30 is a diagrammatic side view showing depression of the plunger of the injection device, thereby effecting injection.

FIG. 31 is a side detail view showing the component of FIG. 26 as the protective cover self-deploys following the injection.

FIG. 32 is a side view similar to FIG. 30, but showing the protective cover in the fully deployed state.

### DETAILED DESCRIPTION

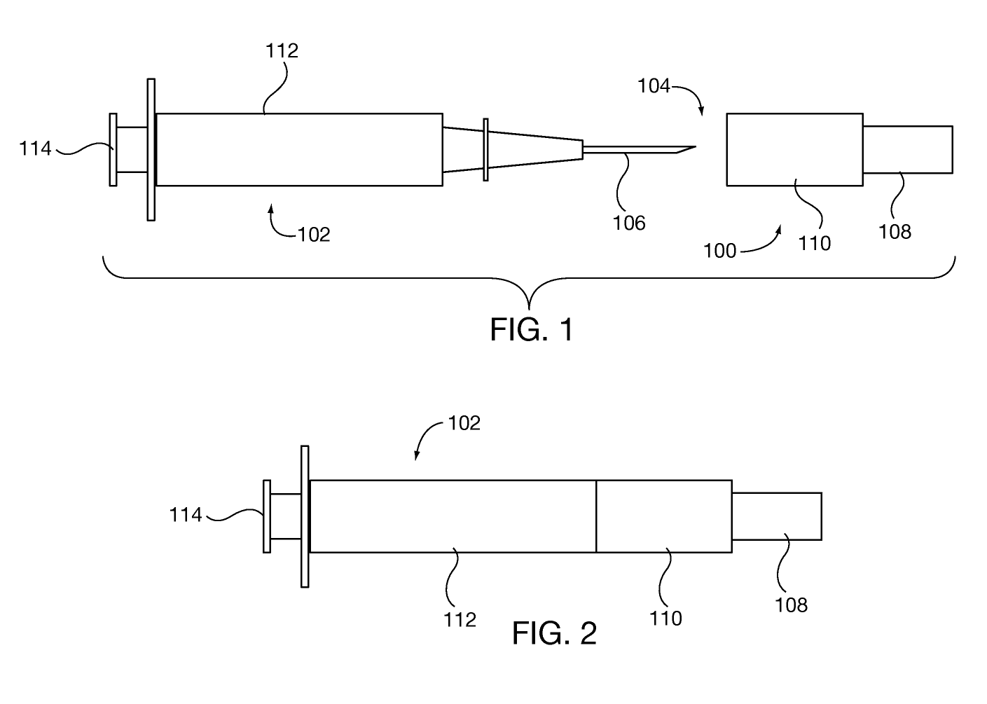

FIGS. 1-23B show some of the contributing operating principles of the present invention, and are the subject of the prior application of record. This subject matter will be presented herein for the convenience of the reader.

FIG. 1 shows a self-deploying protective cover 100 which is separate from and installable onto an injection device such as a syringe 102, such that the protective cover 100 protects personnel (not shown from the point 104 of the needle 106 of the syringe 102. The protective cover comprises two relatively movable parts: a protective sleeve 108 for covering the needle 106, and a stationary base 110 which engages the syringe 102 and remains stationary relative to the syringe 102. The base 110 may be dimensioned and configured to supportably engage the syringe 102. That is, the base 110 fits to or is connected to the syringe 102 and is supported thereon.

FIG. 2 shows the base 110 fit to the syringe 102. In the position shown, it will be observed that the needle 106 is contained within the base 110 and the sleeve 108, and thus is not capable of pricking medical personnel and others. The syringe 102 may be conventional, having a housing 112, a liquid reservoir (not shown) contained within the housing 112 which is in fluid communication with the needle 106, and a plunger 114 for pressurizing fluid within the liquid reservoir for injection purposes. The base 110, shown only representatively in FIGS. 1 and 2, serves as a guide which is disposed to constrain the sleeve 108 to slide along the axis of the needle 106 between an extended position which covers the needle 106, as shown in FIG. 1, and a retracted position which exposes the needle 106 for injection purposes.

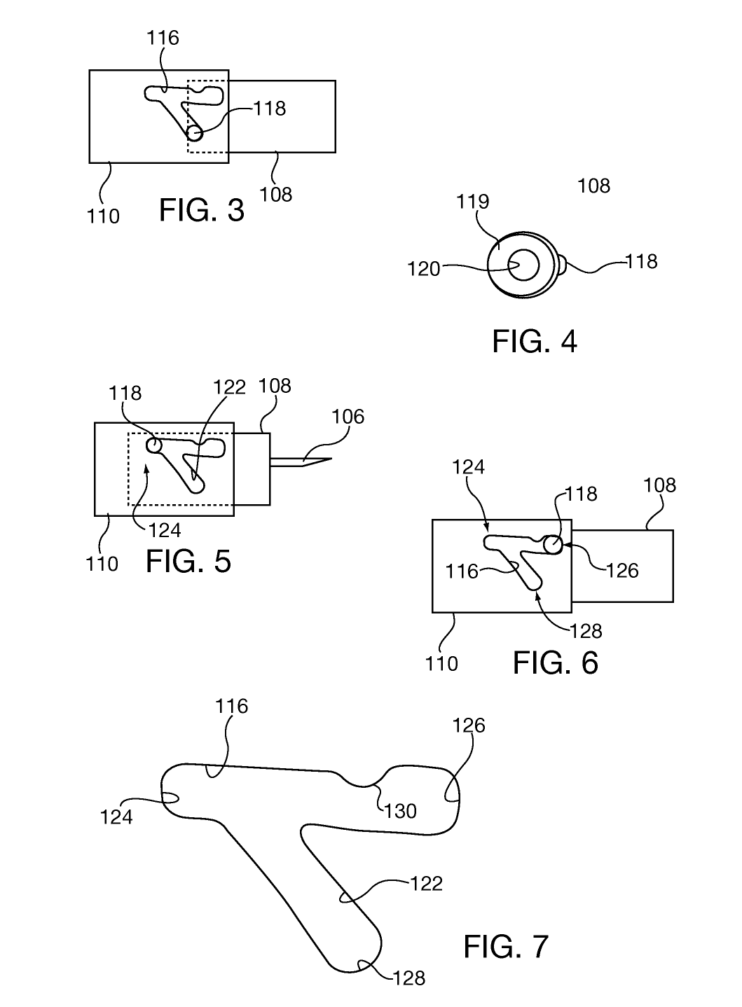

Turning to FIG. 3, the base 110 is seen to be dimensioned and configured to comprise a generally Y-shaped groove 116 formed in the base 110. The exact configuration of the groove 116 is not critical. Therefore, description of the groove 116 as being generally Y-shaped is a semantic convenience. The groove 116 may be V-shaped, or still otherwise shaped, provided that the functions described herein be satisfied by the selected configuration. A projection 118 projects from the sleeve 108, and rides inside the groove 116. The sleeve 108 and its associated projection 118 are seen in end elevation in FIG. 4. The sleeve 108 has an end wall 119 which bears an opening 120 for passing the needle 106 (see FIG. 1).

Cooperation between the sleeve 108 and the base 110 due to interaction between the groove 116 and the projection 118 assures the following motions.

FIG. 3 depicts an initial position of the sleeve 108, wherein the needle 106 is covered, and wherein an injection device such as the syringe 102 to which the base 110 and sleeve 108 are mounted is prepared for an injection. Guiding action occurs responsively to axial pressure imposed on the sleeve by depression of the plunger 114 as the syringe 102 is used to inject fluids.

When the plunger 114 is depressed, a cam surface 122 of the groove 116 acts on the projection 118, forcing the projection 118 to move to the left, as seen in FIG. 5, such that the sleeve 108 is carried along and also moves to the left, thereby exposing the needle 106. In FIG. 5, the projection 118 is shown moved fully to the left, where it comes to seat in one end 124 of the groove 116. Accordingly, the sleeve 108 is moved fully to the left to what may be called the retracted position. The sleeve 108 may inconsequentially rotate or move helically as the projection 118 follows the groove 116.

When the injection is finished, the sleeve 108 is released to return to the extended position. However, rotation or helical motion of the sleeve 108 assures that the projection 118 will come to occupy the other available end 126 of the Y-shaped groove 116, and not that end 128 it occupied in the preparatory stage shown in FIG. 3. This is seen in FIG. 6.

Referring now to FIG. 7, an important distinction between the ends or groove terminals 126 and 128 is that whereas the end 128 cooperates closely enough to guide the projection 118, it does not retain the projection 118. By contrast, the end 126 of the groove 116 does retain the projection 118 due to a bulge 130 which is configured, dimensioned, and located so as to constrict the groove 116 proximate the end or groove terminal 126. The bulge 130 serves as a latch which releasably secures the projection 118 and hence the sleeve 108 in the extended position. Configuration of the bulge 130 is selected in view of the selected constituent material of the sleeve 108 such that the projection 118 may be released from entrapment at the groove terminal 126 by manual pressure, but does not easily or spontaneously become released. This assures that once the projection 118 enters the groove terminal 126, it becomes entrapped, and releasably locks the sleeve 108 in the extended position.

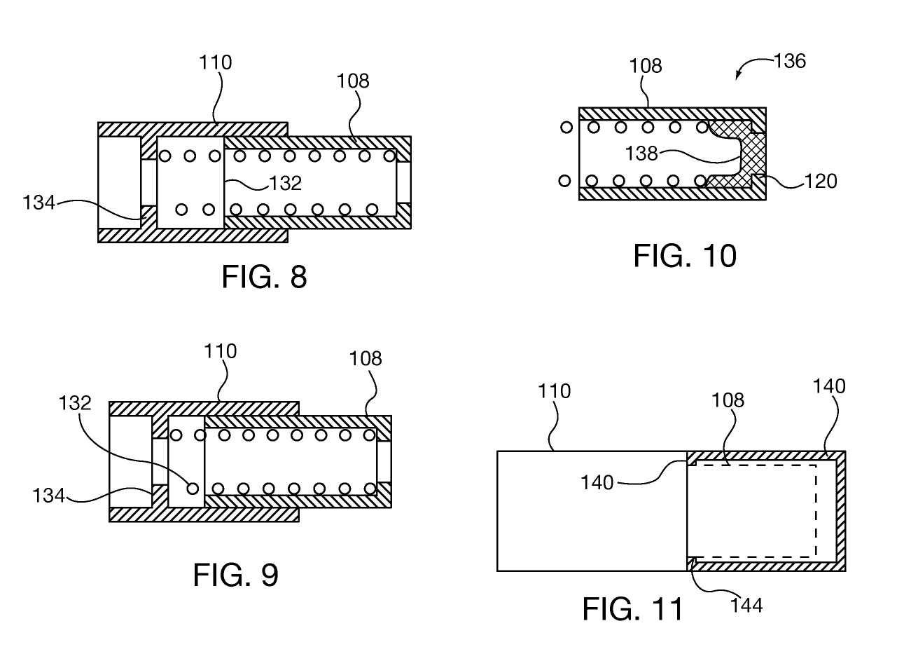

Turning now to FIGS. 8 and 9, it is seen that a spring 132 is placed within the base 110 such that it bears against the sleeve 108. An internal flange 134 provides a seat against which the spring 132 may push. The spring 132 is disposed to urge the sleeve 108 into the extended position. In FIG. 8, the sleeve 108 is shown in the extended position, as would occur in the condition shown in FIG. 3 and also that shown in FIG. 5. In FIG. 9, the sleeve 108 is shown in the retracted position, as also depicted in FIG. 4, with the spring 132 being compressed.

The groove 116 and the spring 132 thus contribute to the sleeve 132 being automatically propelled to the extended, protected position. Therefore, protection ensues following only two actions by the user. In the first action, and starting from the position shown in FIG. 3, where protection from the point 104 of the needle 106 is provided, the user need only depress the plunger 114 and then release the plunger 114. Depression retracts the sleeve 108 to expose the needle 106 for operability. Release enables the spring 132 to return the sleeve to the extended or protected position. The protective feature provided in the present invention in all of its forms may be said to be self-deploying, especially in light of automatic protection of the needle point upon withdrawal of the needle from tissue being injected. This may be contrasted with prior art devices, in which a separate, intentional, additional action is necessary to cover an otherwise dangerously exposed needle point.

A further characteristic of the protective cover 100 is that the limited degree of exposure of the needle 106 assists in limiting and controlling the depth of penetration of the needle 106 into the tissue of the patient such that depth of penetration becomes uniform from one injection to the next.

Configuration of the groove 116 plays a role in that the V described by the groove 116 has two separated groove terminals 126, 128 which of course face the same direction as the needle 106. The projection 118 occupies that groove terminal 128 which does not have a latch, and therefore may readily be moved by manual force into the groove terminal 124. Once the plunger 114 is released, spring action then moves the sleeve 108 linearly and parallel to the needle 106 along that leg of the Y formed by the groove 116 which is parallel to the needle 106. The spring 132 can easily move the projection 118 and hence the sleeve 108 since no twisting or helical motion is needed to travel within that leg of the Y which is parallel to the needle 106. In summary, one end of the V has two separated groove terminals 126, 128 which face the same direction as the travel of the needle 106 during injection, and one end of the Y forming the single point of the Y has one groove terminal 124 and faces oppositely the two separated groove terminals 126, 128. The projection 118 may, prior to injecting, occupy that groove terminal 128 of the two which does not have the latch, but after injection comes to occupy that groove terminal 126 which does have the latch.

Referring now to FIG. 10, a further protective feature is shown. The sleeve 108 may have at its distal end 136, or that end bearing the opening 120 for passing the needle 106, a resilient seal 138 which closes the distal end 136. The resilient seal 138 may be for example a plug or membrane formed from a material such as silicone rubber. The needle 106 can readily pierce and penetrate the resilient seal 138, which seal 138 then closes over the opening left by the needle 106 as the latter withdraws following completion of an injection due to resilience of the constituent material.

FIG. 11 shows a still further protective feature. A cap 140 which is dimensioned and configured to engage the base 110 and to enclose the sleeve 108 assures sterility until it is removed prior to an injection. The cap 140 may be formed with a ring portion 142 which encircles a part of the base 110 or otherwise engages the base 110 so as to resist rotation. A frangible scored junction 144 is located between the ring portion 140 and the cap 140, so that the latter may be twisted or otherwise manually manipulated to break the connection, and thus be removed to expose the sleeve 108. The removed cap 140 may be discarded.

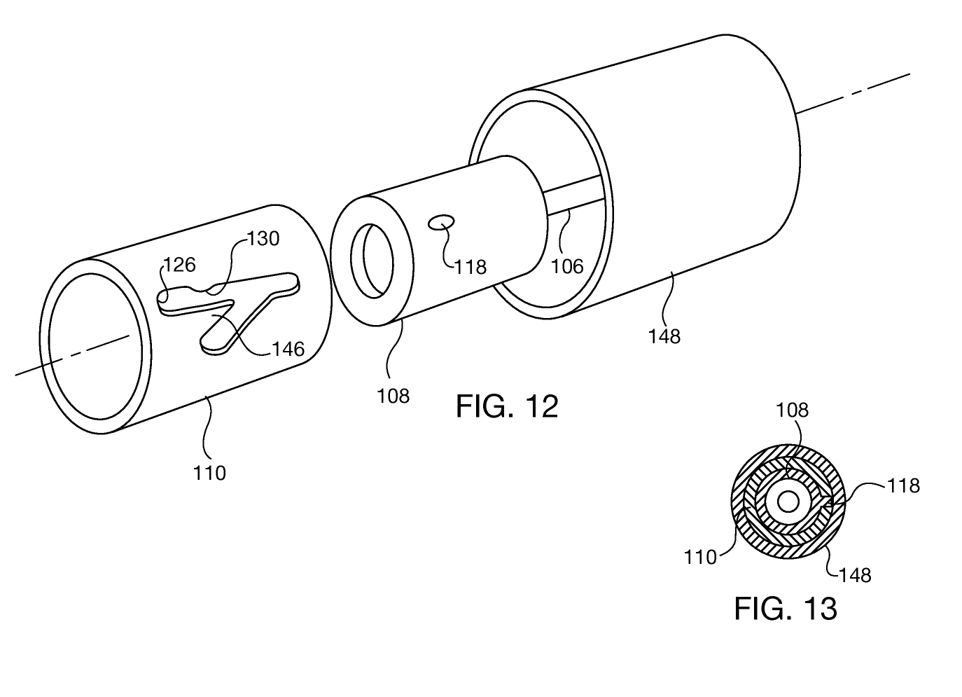

FIG. 12 shows a specific construction which may be utilized. It will be appreciated that the projection 118 may cause some distortion to the bulge 130 and to the center 146 of the Y shape as it is forced into the groove terminal 126. To prevent distortion to the point of inoperability, a reinforcing tube 148 may be provided. The reinforcing tube 148 may be dimensioned and configured to surround and enclose the sleeve 108 along the length of the sleeve 108. The tube 148 may thus be disposed to oppose outward deformation of the sleeve 108 when the protective cover 100 is in use. FIG. 13 shows the close cooperation among the sleeve 108, the base 110, and the tube 148 after they are assembled together.

The operative principles of the groove 116 and the projection 118 may be called on to provide protection similar to that of the arrangement of FIGS. 1-9, but wherein a needle is withdrawn rather than a protective sleeve being extended and retracted.

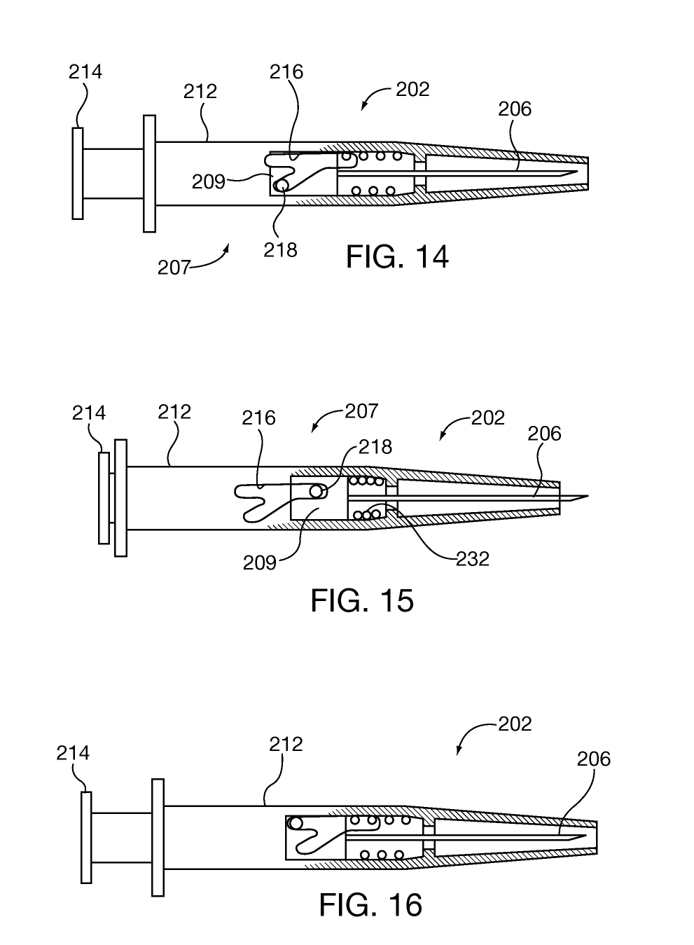

Referring now to FIG. 14, there is shown an injection device such as a syringe 202 having a needle 206 for administering injections, and a self-deploying guard feature for covering the needle 206 which causes the needle to retract automatically such that it withdraws into the protection of a housing 212 of the syringe 202. The syringe 202 may further comprise a fluid reservoir (not seen in FIG. 14) for storing fluid to be injected, disposed within the housing 212, a plunger 214 supported by the housing 212, disposed to effect injection, and a needle assembly 207 disposed in fluid communication with the fluid reservoir and in operable relation to the plunger 214. The needle assembly 207 will be understood to comprise the needle 206 and also a carriage 209, which is a structure for slidably supporting the needle 206 within the housing 212 and for guiding the needle 206 to move in a straight line so that injection may be controlled to penetrate bodily tissue exactly where desired.

The syringe 202 may comprise a self-deploying protective operator disposed to move the needle assembly 207 to an exposed position relative to the housing 212, for administering injections, and to a retracted position relative to the housing 212, wherein the needle 206 is received entirely within the housing 212 and is covered thereby.

The protective operator may comprise a base (not separately shown) which is generally a functional counterpart of the base 110 of FIG. 1. To this end, the base of the syringe 202 is dimensioned and configured to supportably engage the housing 212 and to form a guide which is disposed to constrain the needle assembly 207 to slide along the axis of the housing 212 between the extended position and the retracted position. The extended position refers to the needle 206, and is shown in FIG. 15. FIGS. 14 and 16 show retracted positions. The workings of the protective operator will not be further detailed as they essentially reproduce the workings of the base 110 and the sleeve 108 of the arrangement of FIG. 1, apart from remarking that as with the arrangement of FIG. 1, the protective operator includes a latch which in the syringe 202 is disposed to releasably secure the needle assembly 207 in the retracted position. By contrast, the latch in the arrangement of FIG. 1 engaged the sleeve 108. The latch may be formed by a groove 216 which may be a functional and structural equivalent of the groove 116 of FIGS. 3-5. The groove 216 may be formed in a base member (not separately shown) which may be a structural and functional counterpart of the base 110 of FIG. 1. The groove 216 may engage a projection 218 formed on the carriage 209. The projection 218 will be understood, apart from being fixed to a different component, to operate as does the projection 118 of FIGS. 3-5. To summarize the operation of the groove 216 and the projection 218 in their capacity as a guide, the projection 218 is dimensioned and configured to ride within the groove 216 and to track configuration of the groove 216, thereby guiding the needle assembly 207 when axial pressure is imposed on the needle assembly 207 from the plunger 214 as the syringe 202 is used to inject. Geometry of the groove 216 may be similar to that of the groove 116.

As with the arrangement of FIG. 1, the syringe 202 may include a spring 232 which is disposed to urge the needle assembly 207 into the retracted position.

As with the sleeve 108, the housing 212 of the syringe 202 may be fitted with a resilient seal (not separately shown) which is similar in structure and function to the resilient seal 138 of FIG. 10. In a similar vein, the syringe 202 may comprise a cap (not separately shown) which is similar in structure and function to the cap 140 of FIG. 11.

The syringe 202 may be assembled using a tube similar in structure and function to the tube 148 of FIG. 12, for the purpose of opposing outward deformation of the guide when the protective operator is in use.

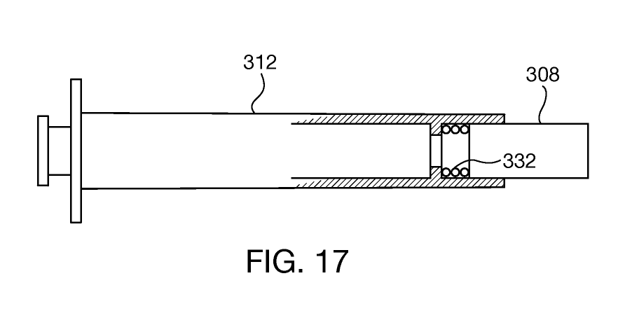

FIG. 17 shows diagrammatically another form of the invention which is similar to the arrangement of FIG. 1, except that the novel protective arrangement is incorporated integrally within an injection device such as a syringe 302. In the syringe 302, that element which corresponds to the base 110 of FIG. 1 is formed integrally with the syringe 302, such as for example, being integral with a housing 312 of the syringe 302. In other respects, the syringe 302 may be similar or identical to the syringe 102 of FIG. 1. For example, the syringe 302 may incorporate a sleeve 308 which is movable under the influence of a plunger 314 and a spring 332 to move to extended and retracted positions as does the sleeve 108 of FIG. 1. The syringe 302 may therefore be summarized as providing an injection device having a protective cover which operates on the principles of the protective cover 100 of FIG. 1.

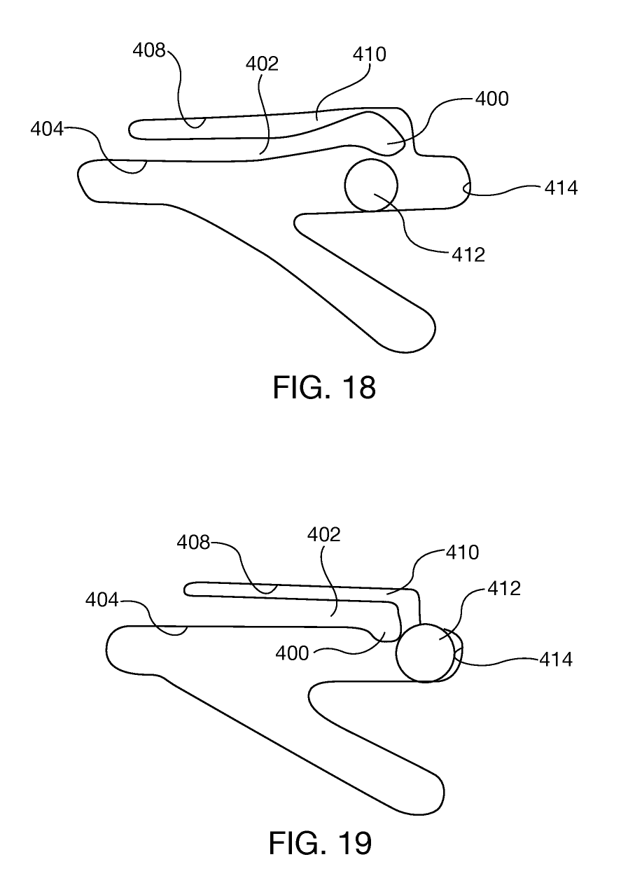

FIGS. 18 and 19 show a variation of the latch arrangement of FIG. 7. In FIG. 7, the bulge 130 is compressed or displaced to enable the projection 118 to pass by. In the arrangement of FIGS. 18 and 19, a corresponding bulge 400 is formed on an arm 402 which is defined by a groove 404. The groove 404 is comparable in purpose to the groove 116, and is formed in a base (not shown per se, but which is comparable in purpose to the base 110) which is configured to define an additional branch 408. The branch 408 provides a relief space 410 which accommodates bending and consequent displacement of the arm 402 as a projection 412, which is comparable in purpose to the projection 118 of FIGS. 3-5, passes by. FIG. 18 shows displacement of the arm 402 as the projection 412 moves to the right. This motion may be caused by depression of a plunger such as the plunger 114 of FIG. 1. However, it must be borne in mind that the arrangement of FIGS. 18 and 19 is equally applicable to an arrangement in which a needle is retracted rather than a protective sleeve being extended, an example of the former being that arrangement shown in FIGS. 14-16.

After the projection 412 has cleared the bulge 400, and as shown in FIG. 19, the arm 402 resumes its normal position, and the projection 412 comes to seat proximate a groove terminal 414 formed in the groove 404.

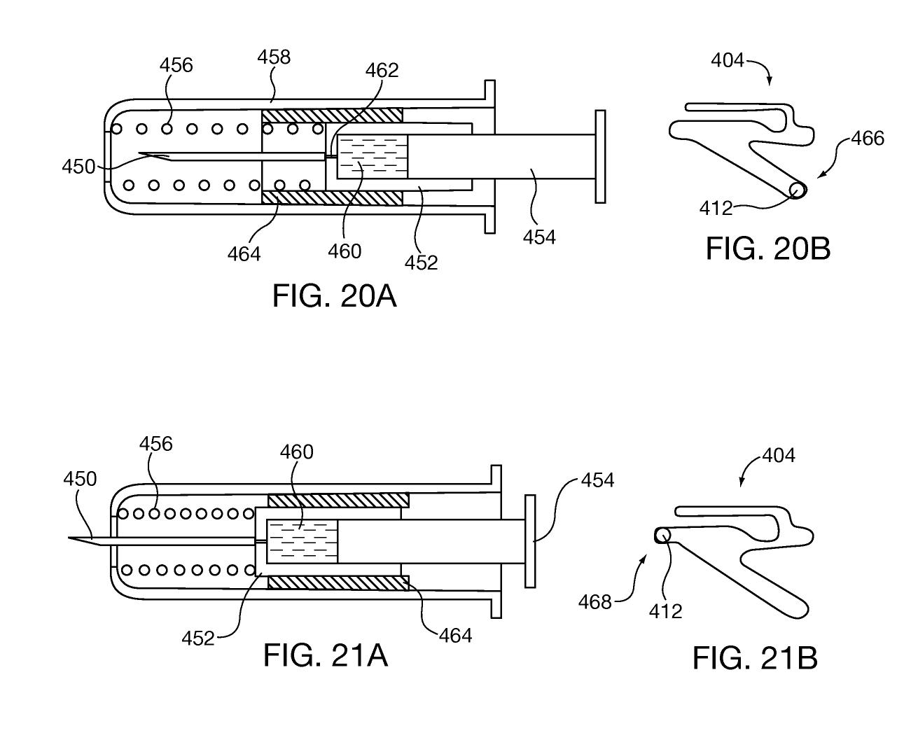

A variation of the arrangement of FIGS. 14-16 is shown in FIGS. 20A, 21A, 22A, and 23A. An initial position of a needle 450, its associated fluid reservoir 452, and an associated plunger 454 are shown in FIG. 20A. A spring 456 is positioned between the fluid reservoir 452, which also serves as a guide for the needle in the way that the carriage 209 performs in FIGS. 14-16, and a stationary outer syringe housing 458. The preparatory condition for an injection as shown in FIG. 20A may be said to constitute a first stage of events.

FIG. 20B shows components of FIG. 20A which cannot be seen in the view of FIG. 20A. An annular groove defining member 464 surrounds the fluid reservoir 452. The projection 412 seen in FIG. 20B may be formed integrally with the fluid reservoir 452, or alternatively could be part of a separate member which would be fixed to the fluid reservoir 452. As explained with respect to FIGS. 18 and 19, the projection 412 rides within and is guided by the groove 404 (shown in both FIGS. 18 and 20B). In the initial stage shown in FIG. 20B, the projection 412 is seated within a groove terminal 466.

FIG. 21A shows a second stage of events which occur as an injection is performed. Initially, depression of the plunger 454 to the left will cause the fluid reservoir 452 to move to the left, thereby advancing the needle 450 until the latter is exposed, and compressing the spring 456. No fluid has been ejected from the fluid reservoir 452 yet as frictional characteristics of the fit of the plunger 454 to the interior surface of the fluid reservoir 452 and of the spring 456, and force required to force fluid through an orifice 462 into the hollow needle 450, are selected to effect operation as described.

At a certain point of travel, the spring is fully compressed. The stationary syringe housing 458 provides a resistive force which causes the fluid reservoir 452 to stop advancing.

Turning momentarily to FIG. 21B, it will be seen that the projection 412 has moved to occupy a groove terminal 468. Because the groove 404 is formed in an annular member, it follows that the fluid reservoir 452 must rotate as it advances, thereby traveling in a helical path. As FIG. 21A is diagrammatic, the needle point is not shown to reflect this rotation.

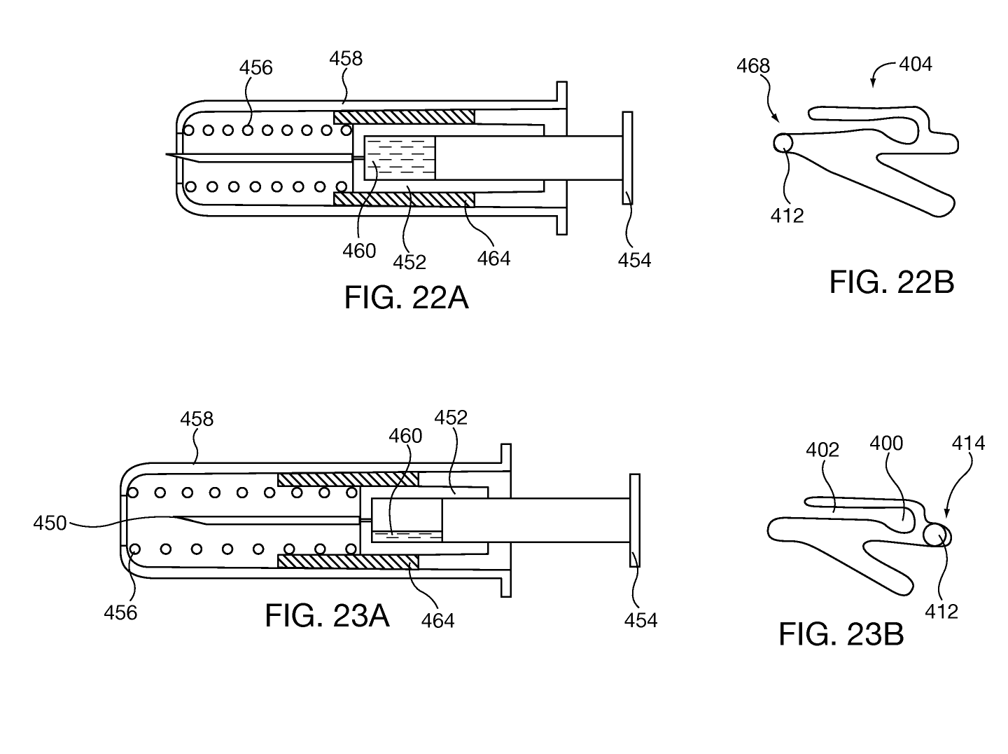

At this point, and as seen in FIG. 22A, continued depression of the plunger 454 will now pressurize fluid 460 contained within the fluid reservoir 452 such that some fluid 460 is ejected from the needle 450. Ejected fluid 460 is depicted as spray for visual effect, but in actual use, for example in medical uses when the needle 450 has penetrated bodily tissue (not shown), no free spray would occur.

Because the fluid reservoir 452 is not moving during this final degree of advance of the plunger 454, the projection 412 does not move, remaining in groove terminal 468. Hence, FIG. 22B is identical to FIG. 21B.

When manual force used to depress the plunger 454 is removed, and referring now to FIG. 23A, the spring 456 will expand and return the fluid reservoir 452 and the needle 450 to their original positions apart from rotation of the fluid reservoir 452 and the needle 450, as seen in FIG. 20 and as repeated in FIG. 23A.

Turning to FIG. 23B, the fluid reservoir 452 and the needle 450 will then be secured in the protected retracted position by the arrangement including the groove 404 and the projection 412. In FIG. 23B, it is seen that the projection 412 has cleared the bulge 400 formed on the arm 402, and thus is locked in position in the groove terminal 414. This arrangement contributes in providing a self-deploying protective operator which is disposed to move the needle assembly (that is, the needle 450 and the fluid reservoir 452, in the embodiment of FIGS. 20A-23A, to an exposed position relative to the syringe housing 458, for administering injections, and then to a retracted position relative to the syringe housing 458, wherein the needle 450 is received entirely within the syringe housing 458 and is covered thereby.

It will be appreciated that many variations and modifications may be introduced to an injection device such as the syringes 102, 202, and 302 without departing from the inventive concepts. For example, a projection such as the projection 118 could be provided on its associated base, such as the base 110, rather than on the mobile component, such as the sleeve 108. Where utilized to effect retraction and extension, springs could be arranged as tension springs rather than compression springs. The latching feature provided for example by the bulge 130 may be caused to operate in a radial direction rather than as described, or may operate in ways other than that shown and described herein. The number of groove terminals may be more than two facing in one direction, as do the terminal ends 126, 128. For example, corresponding groove legs could be provided continuously about a cylindrical member such as the base 110, with a projection such as the projection 118 traveling from one groove leg to a new groove leg each time the associated injection device is used. Springs such as the spring 132 may be embedded or otherwise anchored rather than entrapped between two members. Components which do not move helically and shown as having cylindrical outer surfaces may have other configurations.

In further variations, the base member, such as the base 110 of FIG. 1, may be separate from that member (not shown) which defines a guiding groove, such as the groove 116 of FIGS. 3-5 or the groove 404 of FIGS. 18 and 19. A plunger, such as the plunger 114 of FIG. 1 or the plunger 454 of FIGS. 20-22, may act directly or indirectly on its associated fluid reservoir, such as the fluid reservoir 452, and on a needle guiding and propelling member such as the carriage 209 of FIGS. 14-16. A fluid reservoir such as the fluid reservoir 452 may be a component which is separate from the needle guiding and propelling member such as the carriage 209.

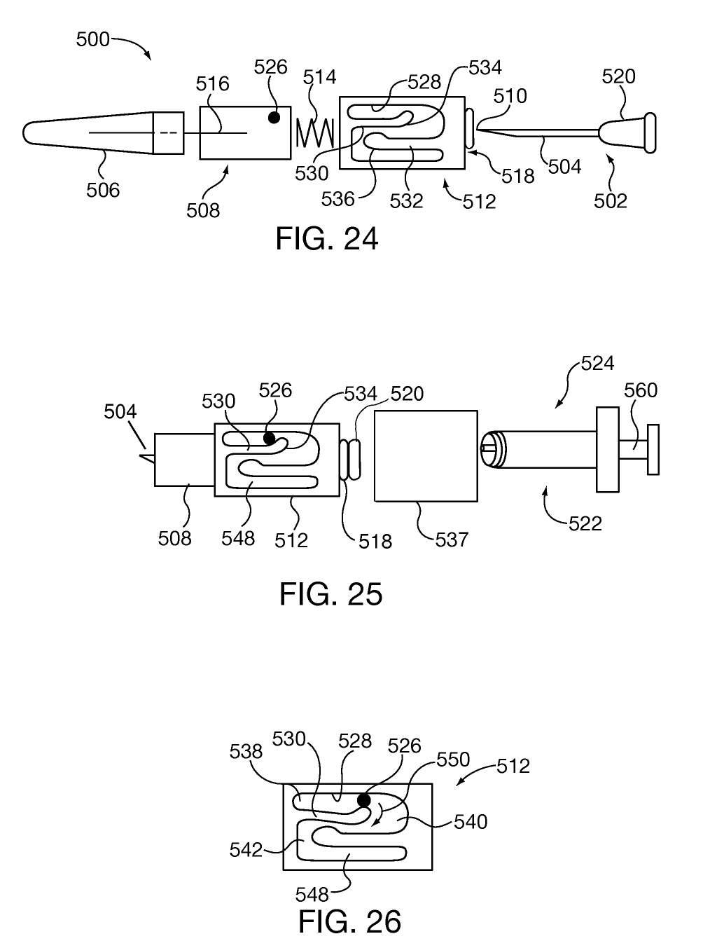

FIG. 24 shows a self-deploying cover 500 which can be affixed to an injection device (seen in its entirety in FIG. 25) having a separate needle assembly 502 having a needle 504. The needle assembly 502 is typical of those provided with a protective cap 506. The self-deploying cover 500 comprises a protective sleeve 508 for covering the point 510 of the needle 504, a base 512 which is dimensioned and configured to supportably engage the needle assembly 502, and a spring 514 the role of which will be described hereinafter. The base 512 may be dimensioned and configured to form a guide which is disposed to constrain the protective sleeve 508 as the protective sleeve 508 slides relative to the base 512 along the axis 516 of the needle 504 as the protective sleeve 508 moves between an extended position in which the protective sleeve 508 covers the needle 504 and a retracted position which exposes the needle 504, for performing injections. The extended position is shown in FIG. 32. Retracted positions are shown in FIGS. 28-30. The spring 514 urges the protective sleeve 508 into the extended position, but this urging force is overcome by manual force to assemble the protective cover 500, for example. A latch arrangement may releasably secure the protective sleeve 508 in the extended position. The base 512 has an attachment element for securing the self-deploying protective cover 500 to the needle assembly 502. This attachment element may take the form of the inner surface of a bore 518 into which the needle assembly 502 is inserted. The needle assembly 502 may be retained by friction.

The cap 506 which is dimensioned and configured to engage the head 520 of the needle assembly 502 by friction for example.

FIG. 25 shows the components of FIG. 24 assembled, and also shows the attachment housing 522 of a standard barrel of a syringe 524 which is used with the needle assembly 502. A needle cap 506 may be used to cover the needle assembly 502. The cap 506 may be removed from the needle assembly 502 once the shot is ready to be administered and ultimately discarded prior to giving injections. The self-deploying cover 500 can be attached to a standard barrel of a syringe 524 by screw fit, friction fit, or any other acceptable method.

Referring momentarily back to FIG. 24, the protective sleeve 508 may have a projection 526 which is intended to ride within and track configuration of a groove 528 formed in the base 512. Cooperation of the projection 526 and groove 528 constrain the protective sleeve 508 to move automatically between the retracted and extended positions. The groove 528 thereby guides the protective sleeve 508 when axial pressure is imposed on the protective sleeve 508 as the syringe is used to inject. The groove 528 is seen to be generally S-shaped. This shape is generated by a first deflectable arm 530 and a second deflectable arm 532 (see FIG. 24). The deflectable arm 530 terminates in a bulge 534. Similarly, the deflectable arm 532 terminates in a bulge 536.

FIG. 25 shows the protective sleeve 508 partly entering the base 512 and exposing the needle 504. In this position, the projection 526 is approaching the bulge 534 of the deflectable arm 530. Continued movement of the protective sleeve 508 into the base 512 will cause the deflectable arm 530 to deflect, bending downwardly as is seen in FIG. 26. The base 512 may be fabricated from an elastic resin, such as nylon or delrin for example, so that its constituent material causes the deflectable arm 530 to yield to the projection 526 as the projection 526 passes, and to return the deflectable arm 530 to its original position after the projection 526 passes. The deflectable arm 532 operates the same way, but differs in its orientation on the base 512.

FIG. 25 also shows a tube 537 which is dimensioned and configured to surround and enclose the elements of the guide feature along the length of the groove 528. The purpose of the tube 537 is purely cosmetic but in additional embodiments can be used to oppose outward deformation of the guide features of the base 512 when the self-deploying protective cover 500 is in use.

Continuing to refer to FIG. 26, the groove 528 is seen to comprise an initial groove terminal 538, an intermediate groove terminal 540, and a final groove terminal 542. These correspond to the initial location of the projection 526 within the groove 528, an intermediate position of the projection 526, as shown in FIG. 29, and a final position of the projection 526, as shown in FIG. 32. These three points also mirror the three ends or terminals of the V-shaped groove of the prior embodiments.

The deflectable arm 530 serves as a first unidirectional gate formed in the groove 528, which is disposed to assure that once the projection 526 has passed the first unidirectional gate as the projection 526 moves from its initial position to its intermediate position in negotiating the groove 528, the projection 526 is constrained from re-assuming a position prior to passing the first unidirectional gate.

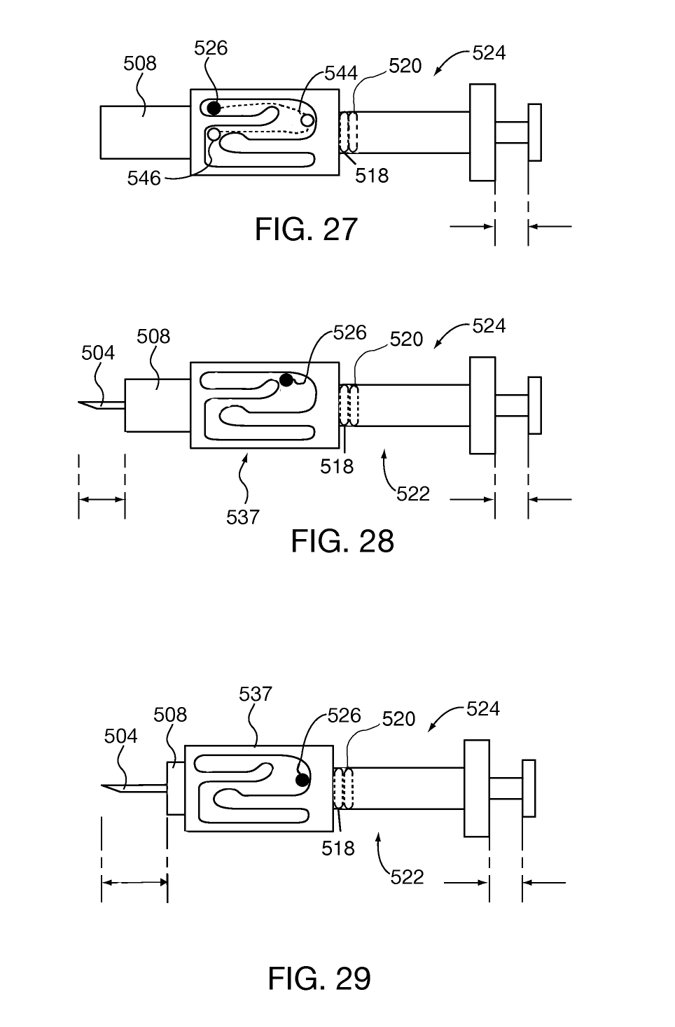

FIG. 27 shows the initial position of the projection 526 and of the protective sleeve 508. In the initial position, the protective sleeve 508 is in the extended position covering the needle 504. FIG. 27 also shows two subsequent positions of the projection 526, these two being rendered in uncolored circles 544, 546. These subsequent positions coincide with intermediate groove terminal 540 and final groove terminal 542. As it is formed, the S-shaped groove 528 has a leg 548 (see FIG. 25). This leg 548 allows space for the deflectable arm 532 to deflect when the projection 526 moves it aside, but serves no further role in providing a path for the travels of the projection 526.

Continued travel of the projection 526 as the protective sleeve 508 moves to the retracted position is shown in FIG. 28. In FIG. 28, the projection 526 is approaching the intermediate groove terminal 540. Effect on the arm 530 is seen in FIG. 26, where the deflectable arm 530 is deflecting in the direction of an arrow 550. When the projection 526 fully clears the deflectable arm 530, the arm 530 will return to the position shown in FIG. 24. The bulge 534 assures that once the projection 526 has passed the deflectable arm 530, which deploys only after the protective sleeve 508 is in the retracted position.

Referring now to FIG. 29, the projection 526 reaches its furthest point of travel, seating within the intermediate groove terminal, when the protective cover 508 fully exposes the needle 504.

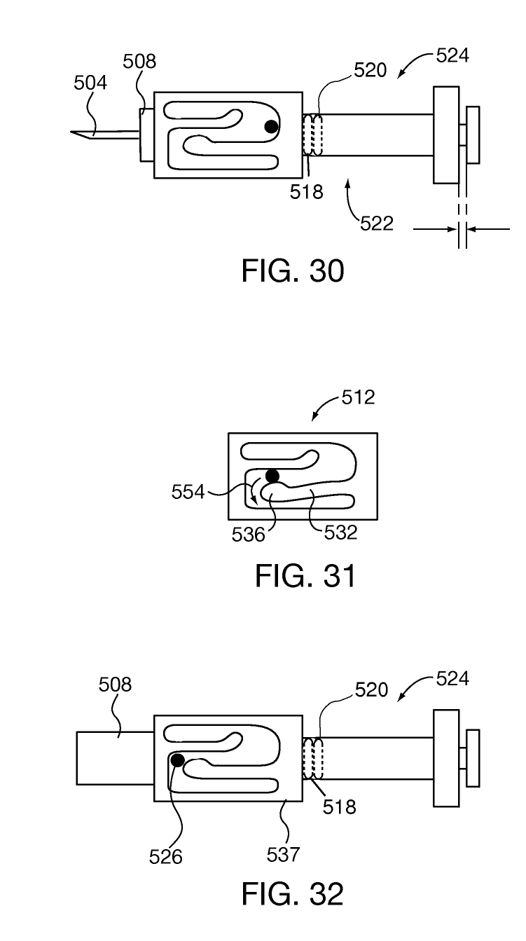

FIG. 30 shows the plunger 560 of the syringe 524 in the depressed condition, thus effecting injection. Depressing the plunger 560 expels liquid stored in the reservoir of the syringe 524, but does not necessarily project the needle 504. The needle 504 has been passed into the body of the patient by moving the entire syringe 524, with the needle 504 exposed. The protective cover 508 can be made in different dimensions and configurations, for example to correspond to different standard syringe dimensions and injection depths. The protective cover 508 can be dimensioned and configured to be received entirely within the base 512 and the tube 537 if desired.

When the injection is complete, the protective cover 508 moves back to the extended position seen in FIG. 32 automatically, urged by the spring 514 (FIG. 24). As this happens, and referring to FIG. 31, the projection 526 passes the bulge 536 of the deflectable arm 532. The deflectable arm 532 deflects as indicated by the arrow 554. The position of full extension of the protective sleeve 508 is shown in FIG. 32, with the projection 526 occupying the final groove terminal 542. The deflectable arm 532 returns to its usual position (shown for example in FIG. 24). The deflectable arm 532 serves as a second unidirectional gate formed in the groove 528, which is disposed to assure that once the projection 526 has passed the second unidirectional gate after the protective sleeve has reached the retracted position of FIG. 30, and upon return of the protective sleeve 508 to the extended position seen in FIG. 32, the protective sleeve 508 is constrained from re-assuming a position prior to passing the second unidirectional gate.

Because the second unidirectional gate entraps the projection 526 with the protective sleeve 508 in the fully extended position covering the needle 504, the second unidirectional gate serves as a latch retaining the protective sleeve 508 in the extended position. Of course, this position can be overcome by manual force, but the protective sleeve 508 is nonetheless retained against casual unintended movement which might otherwise expose the needle 508. This occurs because the bulge 536 is dimensioned and configured to constrict the groove 528 proximate the bulge 536, thereby denying return passage to the projection 526.

It should be observed that the present invention may be thought of as the self-deploying protective cover 500 or alternatively as an injection device such as the syringe 524 having such a self-deploying protective cover 500.

The invention may also be thought of as a self-deploying protective cover comprising a protective sleeve such as the protective sleeve 508, a base such as the base 512, and a spring disposed to urge the protective sleeve into an extended position for covering a needle, such as the spring 514, the self-deploying protective cover comprising a first unidirectional gate formed in a groove such as the groove 528, and a second unidirectional gate formed in the groove.

The various embodiments presented herein have been described as employing friction fit for connections, such as connecting the base 512 to the needle assembly 502, and connecting the protective cap 506 to the protective sleeve 508. Optionally, shoulders (not shown) are formed in the protective sleeve 508 and in the base 512 to prevent overtravel of any of these components by interference. Connections other than by friction are contemplated as being within the scope of the invention. For example, threaded connections, bayonet connections, resiliently compressible detents, pins and aligned holes, other forms of connections (none shown) or any combination of such connection elements may be substituted for the friction connection to join any of the components presented herein where feasible.

It would also be possible to cause the base of the protective cover engage the housing of the injection device, instead of or in addition to engaging the needle assembly.

Action of deflectable members such as the deflectable arms such as the deflectable arms 530 and 532 for example may be modified such that the displacement occurs in a radial direction relative to the axis of the needle of the injection device, such as the needle 504. Where such modification is practiced, elements such as the tube 537 may have recesses to accommodate this displacement but to continue to oppose outward deformation in areas not associated with deflection.

While the present has been described in connection with what is considered the most practical and preferred embodiments, it is to be understood that the present invention is not to be limited to the disclosed arrangements, but is intended to cover various arrangements which are included within the spirit and scope of the broadest possible interpretation of the appended claims so as to encompass all modifications and equivalent arrangements which are possible.

What is claimed is:

1. A self-deploying protective cover which can be affixed to an injection device having a separate needle assembly having a needle, so as to selectively cover and expose the needle, comprising:
a protective sleeve for covering the needle of the injection device; and
a base which is dimensioned and configured to supportably engage the needle assembly and which is dimensioned and configured to form a guide which is disposed to constrain the protective sleeve to slide along the axis of the needle between an extended position which covers the needle and a retracted position which exposes the needle;
a spring which is disposed to urge the protective sleeve into the extended position,
a latch disposed to releasably secure the protective sleeve in the extended position; and
an attachment element for securing the self-deploying protective cover to the separate needle assembly.

2. The self-deploying protective cover of claim 1, wherein the guide comprises a groove formed in the base, having an initial groove terminal, an intermediate groove terminal, and a final groove terminal.

3. The self-deploying protective cover of claim 2, wherein the guide comprises a projection formed in the protective sleeve, which said projection is dimensioned and configured to ride within the groove and to track configuration of the groove, thereby guiding the sleeve when axial pressure is imposed on the sleeve as the injection device is used to inject.

4. The self-deploying protective cover of claim 3, further comprising a first unidirectional gate formed in the groove which is disposed to assure that once the projection has passed the first unidirectional gate as the projection negotiates the groove, the projection is constrained from re-assuming a position prior to passing the first unidirectional gate.

5. The self-deploying cover of claim 4, wherein the first unidirectional gate deploys only after the sleeve is in the retracted position.

6. The self deploying cover of claim 3, further comprising a second unidirectional gate formed in the groove which is disposed to assure that once the projection has passed the second unidirectional gate after the protective sleeve has reached the retracted position and upon return of the protective sleeve to the extended position, the protective sleeve is constrained from re-assuming a position prior to passing the second unidirectional gate.

7. The self-deploying protective cover of claim 4, wherein the first unidirectional gate comprises a deflectable arm which is deflected by the projection when the projection passes the deflectable arm as the projection rides within the groove.

8. The self-deploying protective cover of claim 6, wherein the latch comprises a bulge which is formed on the second unidirectional gate, and which is dimensioned and configured to constrict the groove proximate the bulge.

9. The self-deploying protective cover of claim 6, wherein the second unidirectional gate serves as the latch.

10. An injection device having a protective cover, comprising:
a housing, a fluid reservoir for storing fluid to be injected, disposed within the housing, and a plunger supported by the housing, disposed to effect injection, the housing having a first attachment element; and
a separate needle assembly which is attachable to the housing and is disposable in fluid communication with the fluid reservoir and in operable relation to the plunger, comprising a needle, structure for slidably supporting the needle within the housing; and
a self-deploying protective cover which is separate from the housing, comprising
a protective sleeve for covering the needle of the injection device,
a base which is dimensioned and configured to supportably engage the needle assembly of the injection device and which is dimensioned and configured to form a guide which is disposed to constrain the protective sleeve to slide along the axis of the needle between an extended position which covers the needle, and a retracted position which exposes the needle, and
a latch disposed to releasably secure the protective sleeve in the extended position, the needle assembly having a second attachment element which is manually engageable with and detachable from the first attachment element.

11. The injection device of claim 10, wherein the guide comprises a groove formed in the base, having an initial groove terminal, an intermediate groove terminal, and a final groove terminal, and a projection formed in the sleeve, wherein said projection is dimensioned and configured to ride within the groove and to track configuration of the groove, thereby guiding the sleeve when axial pressure is imposed on the sleeve as the injection device is used to inject.

12. The injection device of claim 11, further comprising a first unidirectional gate formed in the groove which is disposed to assure that once the projection has passed the first unidirectional gate as the projection negotiates the groove, the projection is constrained from re-assuming a position prior to passing the first unidirectional gate.

13. The injection device of claim 12, wherein the first unidirectional gate deploys only after the sleeve is in the retracted position.

14. The injection device of claim 11, further comprising a second unidirectional gate formed in the groove which is disposed to assure that once the projection has passed the second unidirectional gate after the protective sleeve has reached the retracted position and upon return of the protective sleeve to the extended position, the protective sleeve is constrained from re-assuming a position prior to passing the second unidirectional gate.

15. The injection device of claim 14, wherein the first unidirectional gate comprises a deflectable arm which is deflected by the projection when the projection passes the deflectable arm as the projection rides within the groove.

16. The injection device of claim 14, wherein the latch comprises a bulge which is formed on the second unidirectional gate, and which is dimensioned and configured to constrict the groove proximate the bulge.

17. The injection device of claim 14, wherein the second unidirectional gate serves as the latch.

18. The injection device of claim 10, further comprising a resilient seal disposed to close that end of the housing from which the needle projects during injections.

19. The injection device of claim 10, comprising a cap which is dimensioned and configured to engage the housing and to enclose that end of the housing from which the needle projects during injections.

20. The injection device of claim 11, further comprising a tube which is dimensioned and configured to surround and enclose the guide along the length of the guide, whereby the tube is disposed to oppose outward deformation of the guide when the self-deploying protective operator is in use.

21. A self-deploying protective cover for the needle of an injection device, comprising:
a protective sleeve for covering the needle of the injection device; and
a base which is dimensioned and configured to supportably engage the injection device and which is dimensioned and configured to form a guide which is disposed to constrain the protective sleeve to slide along the axis of the needle between an extended position which covers the needle and a retracted position which exposes the needle, wherein the guide comprises a groove formed in the base, having
an initial groove terminal, an intermediate groove terminal, and a final groove terminal, and a projection formed in the sleeve, wherein said projection is dimensioned and configured to ride within the groove and to track configuration of the groove, thereby guiding the sleeve when axial pressure is imposed on the sleeve as the injection device is used to inject,
a first unidirectional gate formed in the groove which is disposed to assure that once the projection has passed the first unidirectional gate as the projection negotiates the groove, the projection is constrained from re-assuming a position prior to passing the first unidirectional gate and attaining the location of the intermediate groove terminal, and wherein the first unidirectional gate deploys only after the sleeve is in the retracted position, and
a second unidirectional gate formed in the groove which is disposed to assure that once the projection has passed the second unidirectional gate after the protective sleeve has reached the retracted position, the projection has attained the final groove terminal, and upon return of the protective sleeve to the extended position, the protective sleeve is constrained from re-assuming a position prior to passing the second unidirectional gate; and
a spring which is disposed to urge the protective sleeve into the extended position.
